(19) United States
(12) Patent Application Publication
(10) Pub. No.: US 2010/0174322 A1
(43) Pub. Date: Jul. 8, 2010

(54) Biased Bumper Mechanism and Method

(75) Inventors: Mahmoud F. Abdelgany, Rockaway, NJ (US); Young Hoon Oh, Montville, NJ (US)

Correspondence Address: Rahman LLC, 10025 Governor Warfield Parkway, Suite 110, Columbia, MD, 21044, US

(73) Assignee: CUSTOM SPINE, INC., Parsippany, NJ (US)

(21) Appl. No.: 12/348,283

(22) Filed: Jan. 3, 2009

(51) Int. Cl. A61B 17/04

(52) U.S. Cl. 606/301; 606/305; 606/308

(57) ABSTRACT

A dynamic screw assembly includes a screw head having a pair of diametrically opposed arms, a slot between the arms, an inwardly curved bottom portion, an outwardly protruding and expandable bulbous end extending from the inwardly curved bottom portion and an opening positioned through the bulbous end, a bumper mechanism adjacent to the screw head that adjusts an angle of the screw head to a desired location in the dynamic screw assembly, a fixation component coupled to the bumper mechanism, a saddle connection positioned in the opening and engaging the screw head and the fixation component, a longitudinal member positioned in the slot and a blocker coupled to the screw head and the longitudinal member.

### BACKGROUND

1. Technical Field

The embodiments herein generally relate to devices used in spinal surgeries, and, more particularly, to a biased bumper mechanism to achieve a desired poly-axial dynamism regardless of the insertion angle of the implant assembly in a dynamic screw system.

2. Description of the Related Art

Dynamic stabilization is a surgical procedure performed to change the biomechanics of the affected lumbar segment by reducing the load on the disc without loss of motion. A dynamic system works by limiting motion and altering stress patterns across the degenerated segment, preventing excessive motion or postures that result in pain. In dynamic spine stabilization, the vertebrae are stabilized while leaving the spine itself intact, and capable of bending, straightening, or twisting within new limits. There are conventional devices that use the biased angle concept. These devices are rigid and fixed for fusion applications. Further, they do not provide for the surgeon to adjust the device to a desired location for a given insertion angle.

### SUMMARY

In view of the foregoing, an embodiment herein provides a dynamic screw assembly. The dynamic screw assembly includes a screw head having a pair of diametrically opposed arms, a slot between the arms, an inwardly curved bottom portion, an outwardly protruding and expandable bulbous end extending from the inwardly curved bottom portion and an opening positioned through the bulbous end, a bumper mechanism adjacent to the screw head that adjusts an angle of the screw head to a desired location in the dynamic screw assembly, a fixation component coupled to the bumper mechanism, a saddle connection positioned in the opening and engaging the screw head and the fixation component, a longitudinal member positioned in the slot and a blocker coupled to the screw head and the longitudinal member.

The bumper mechanism may include any of a one-piece bumper and a stacked bumper. The one-piece bumper may generate a resultant angle, the resultant angle is any of a zero degree angle or a sum of an angle by the one-piece bumper and the fixation component. The stacked bumper may generate a resultant angle and the resultant angle is an accumulated angle between the stacked bumper and the fixation component. The bumper mechanism may limit an angulation of the screw head based on an orientation of the bumper mechanism with respect to the fixation component.

The fixation component includes an open concave head and a threaded end. The open concave head of the fixation component may contact the bumper mechanism. The open concave head of the fixation component includes an inner portion that receives the bulbous end of the screw head, a hole and an outer portion comprising grooves. The bulbous end may be positioned opposite to the pair of diametrically opposed arms. The hole of the fixation component preferably engages the saddle connection.

Another embodiment provides an apparatus for dynamic spinal stabilization. The apparatus includes at least one bumper having a flexible material and composed of two intersecting planes, the bumper adjusts an insertion angle of the apparatus to a desired location based on an orientation of the bumper, a bone anchor having an open concave head and a threaded end, the open concave end engages the bumper, a coupling member having a first portion including a pair of arms that are diametrically opposed, a U-shaped slot positioned between the pair of arms, an inwardly curved bottom portion, a second portion having a an outwardly protruding and expandable bulbous end extending from the inwardly curved bottom portion configured to engage the open concave head of the bone anchor and an opening positioned between the first portion and the second portion, a saddle connection that engages the opening of the coupling member, the saddle connection being coupled to the bone anchor, a rod coupled to the U-shaped slot and a threaded blocker that engages the pair of arms of the coupling member and secures the rod in the coupling member.

The open concave head of the bone anchor further includes an inner portion that receives the bulbous end of the coupling member, a hole that engages the saddle connection and an outer portion comprising grooves. The pair of arms includes an outer wall and an inner wall, the outer wall having an indent feature and the inner wall having threads. The bumper preferably includes any of at least a one-piece bumper and a stacked bumper. The stacked bumper includes a slot and generate a resultant angle. The resultant angle is an accumulated angle between the stacked bumper and the bone anchor. The one-piece bumper may generate a resultant angle. The resultant angle is any of a zero degree angle or a sum of an angle by the one-piece bumper and the bone anchor.

Yet another embodiment provides a method of inserting a dynamic screw assembly in a vertebral body. The method includes engaging the dynamic screw assembly with the vertebral body, the dynamic screw assembly includes a screw head having a pair of diametrically opposed arms, a slot between the arms, an inwardly curved bottom portion, an outwardly protruding and expandable bulbous end extending from the inwardly curved bottom portion and an opening positioned through the bulbous end, a bumper mechanism adjacent to the screw head that adjusts an angle of the screw head to a desired location in the dynamic screw assembly, a fixation component coupled to the bumper mechanism, a saddle connection positioned in the opening and engaging the screw head and the fixation component, a longitudinal member positioned in the slot and a blocker coupled to the screw head and the longitudinal member, positioning the fixation component to form a first angle, adjusting a top portion of the bumper mechanism to form a second angle and obtaining a resultant angle between the fixation component and the bumper mechanism.

The resultant angle is any of a zero degree angle or a summation of the first angle and the second angle. The resultant angle may be obtained based on an orientation of the bumper mechanism being stacked. The resultant angle is an accumulation of the first angle of the bumper mechanism and the second angle of the fixation component. The stacked bumper mechanism may generate a resultant angle. The resultant angle may be an accumulated angle between the stacked bumper mechanism and the fixation component.

The bumper mechanism may limit an angulation of the screw head based on an orientation of the bumper mechanism with respect to the fixation component. The fixation component includes an open concave head and a threaded end. The open concave head of the fixation component preferably contacts the bumper mechanism. The open concave head of the fixation component includes an inner portion that receives the bulbous end of the screw head, a hole and an outer portion including grooves. The bulbous end is positioned opposite to the pair of diametrically opposed arms. The hole of the fixation component preferably engages the saddle connection.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIGS. 1A through 1C illustrate assembled front views of a dynamic screw assembly in a first position, a second position, and a third position, respectively, according to an embodiment herein;

FIGS. 2A through 2C illustrate cross-sectional views of the dynamic screw assembly of FIGS. 1A through 1C according to an embodiment herein;

FIGS. 3A and 3B illustrate a perspective view and a front view, respectively, of the bone anchor of the dynamic screw assembly of FIGS. 1A through 1C according to an embodiment herein;

FIGS. 3C and 3D illustrate cross-sectional views of the bone anchor of the dynamic screw assembly of FIG. 3B in a first position and a second position, respectively, according to an embodiment herein;

FIGS. 3E and 3F illustrate top views of the bone anchor of the dynamic screw assembly of FIG. 3B in the first position and the second position, respectively, according to an embodiment herein;

FIGS. 4A through 4D illustrate a perspective view, a front view, a cross-sectional view, and a top view, respectively, of the coupling member of the dynamic screw assembly of FIGS. 1A through 1C according to an embodiment herein;

FIGS. 5A through 5C illustrate a perspective view, a front view, and a top view, respectively, of a rod of the dynamic screw assembly of FIGS. 1A through 1C according to an embodiment herein;

FIGS. 6A through 6D illustrate a perspective view, a front view, a top view, and a cross-sectional view, respectively, of the blocker of the dynamic screw assembly of FIGS. 1A through 1C according to an embodiment herein;

FIGS. 7A through 7C illustrate a perspective view, a front view, and a top view, respectively, of the saddle connection of the dynamic screw assembly of FIGS. 1A through 1C according to an embodiment herein;

FIGS. 8A through 8D illustrate a perspective view, a front view, a top view, and a cross-sectional view, respectively, of the biased bumper of the dynamic screw assembly of FIGS. 1A through 1C according to a first embodiment herein;

FIGS. 9A through 9D illustrate a perspective view, a front view, a cross-sectional view, and a top view, respectively, of the biased bumper of the dynamic screw assembly of FIGS. 1A through 1C according to a second embodiment herein;

FIGS. 10A through 10D illustrate a perspective view, a front view, a cross-sectional view, and a top view, respectively, of the biased bumper of the dynamic screw assembly of FIGS. 1A through 1C according to a third embodiment herein;

FIGS. 11A through 11D illustrate a perspective view, a front view, a cross-sectional view, and a top view, respectively, of the biased bumper of the dynamic screw assembly of FIGS. 1A through 1C according to fourth embodiment herein; and

FIG. 12 is a flow diagram illustrating a method according to an embodiment herein.

### DETAILED DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As indicated above, there remains a need for a dynamic screw assembly which can be fixed to a desired position later during the surgery. The embodiments herein achieve this by providing a biased bumper mechanism which assists the surgeon to adjust the angulation of the coupling member to a desired location in a dynamic screw implant. With the help of the bumper mechanism, the bone anchor can be inserted in any direction at the time of implanting the assembly and later be adjusted to a final desired position.

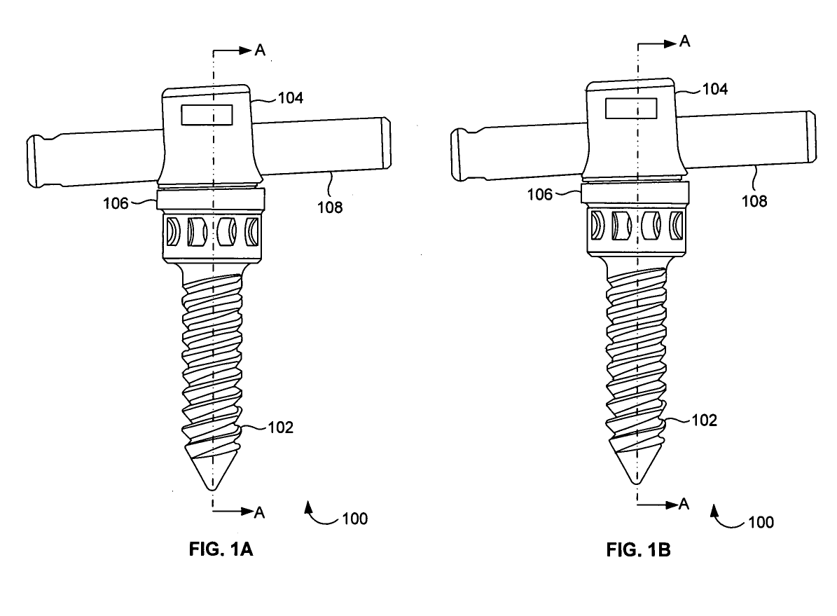

Thus, the biased bumper mechanism helps the surgeon to achieve a desired polyaxial dynamism regardless of the insertion angle of the implant assembly. Referring now to the drawings, and more particularly to FIGS. 1A through 12, where similar reference characters denote corresponding features consistently throughout the figures, there are shown embodiments of the invention.

FIGS. 1A through 1C illustrate assembled front views of a dynamic screw assembly 100 in a first position, a second position, and a third position, respectively, according to an embodiment herein. The dynamic screw assembly 100 includes a bone anchor 102, a coupling member 104, biased bumper(s) 106, and a rod 108. The bone anchor 102 may be a fixation component to be inserted into the bone (not shown). The top portion of the bone anchor 102 may be angled to accept the bumper(s) 106. The coupling member 104 may be embodied as a screw head connecting the bone anchor 102 and the rod 108.

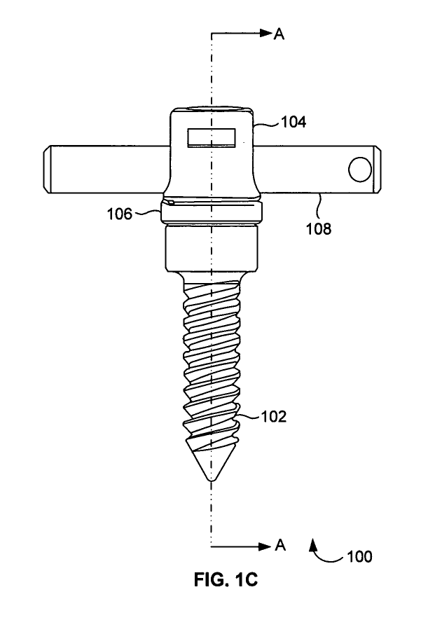

The biased bumper(s) 106 may be located between the bone anchor 102 and the coupling member 104. The biased bumper(s) 106 may provide a mechanism for adjusting the angulation of the coupling member 104 to a desired angle in the dynamic screw assembly 100 and allows for the fixation of the bone anchor 102 to a desired location after implanting the dynamic screw assembly 100 in the spine (not shown). The rod 108 may be embodied as a longitudinal member positioned along a horizontal axis in the coupling member 104 to connect a saddle connection 202 (shown in FIGS. 2A through 2C).

FIGS. 2A through 2C illustrate cross-sectional views of the dynamic screw assembly 100 of FIGS. 1A through 1C according to an embodiment herein. The dynamic screw assembly 100 includes a saddle connection 202, a blocker 204, the bone anchor 102, the coupling member 104, the biased bumper(s) 106, and the rod 108. The saddle connection 202 may be placed along a vertical axis through the center of the coupling member 104 to prevent the coupling member 104 from disengaging the bone anchor 102 and limit angulation. The blocker assembly 204 may be the securing member between the rod 108 and the coupling member 104 and pushes down onto the saddle connection 202 to effectively lock the dynamic screw assembly 100.

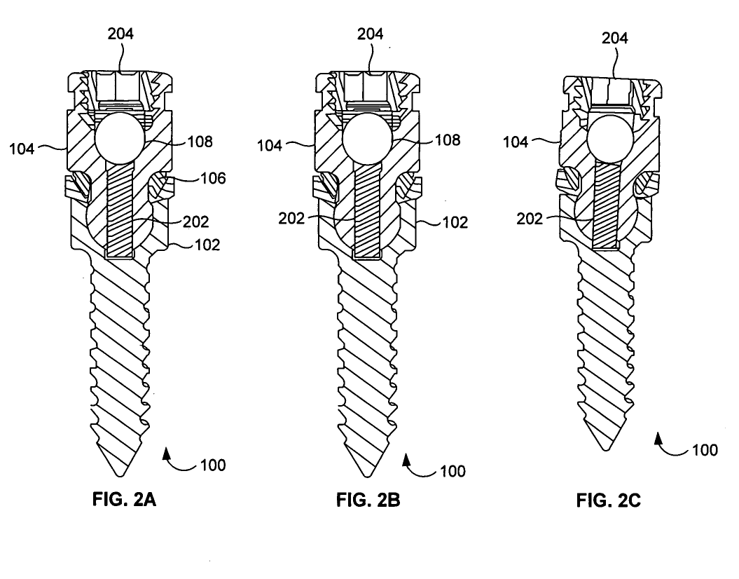

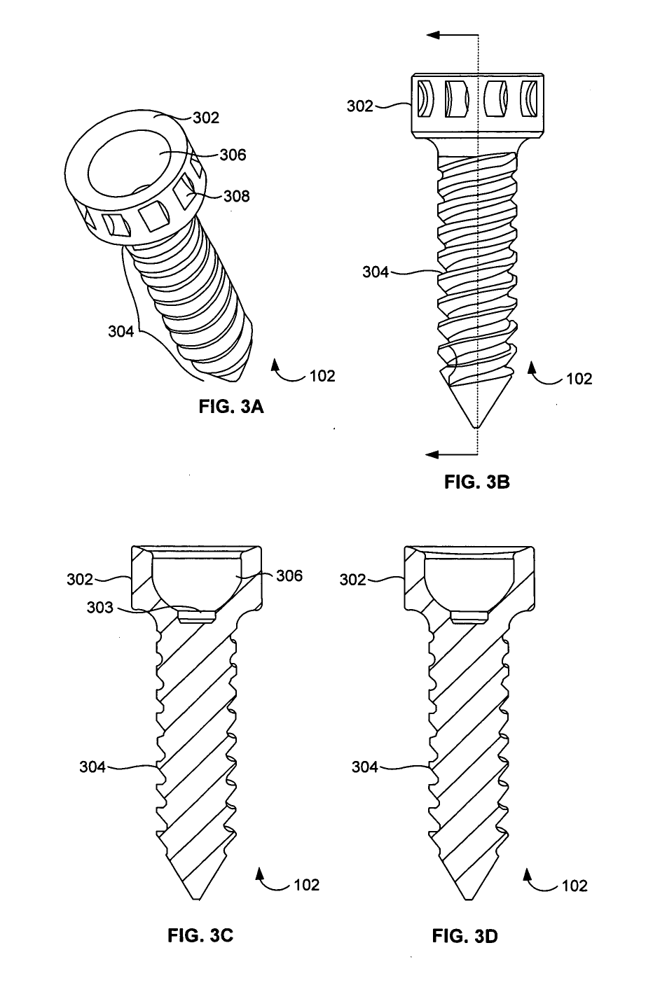

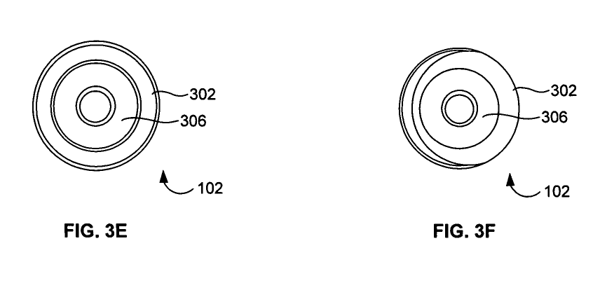

FIGS. 3A and 3B illustrate a perspective view and a front view, respectively, of the bone anchor 102 of the dynamic screw assembly 100 of FIGS. 1A through 1C according to an embodiment herein. The bone anchor 102 includes an open concave head 302, a threaded portion 304. The open concave head 302 further includes an inner portion 306 and grooves 308. The inner portion 306 of the open concave head 302 receives the bulbous end 406 of the coupling member 104 and the saddle connection 202 (of FIGS. 2A through 2C), wherein the saddle connection 202 rests in a small hole 303. FIGS. 3C and 3D illustrate cross-sectional views of the bone anchor 102 of the dynamic screw assembly 100 of FIG. 3B in a first position and a second position, respectively, according to an embodiment herein.

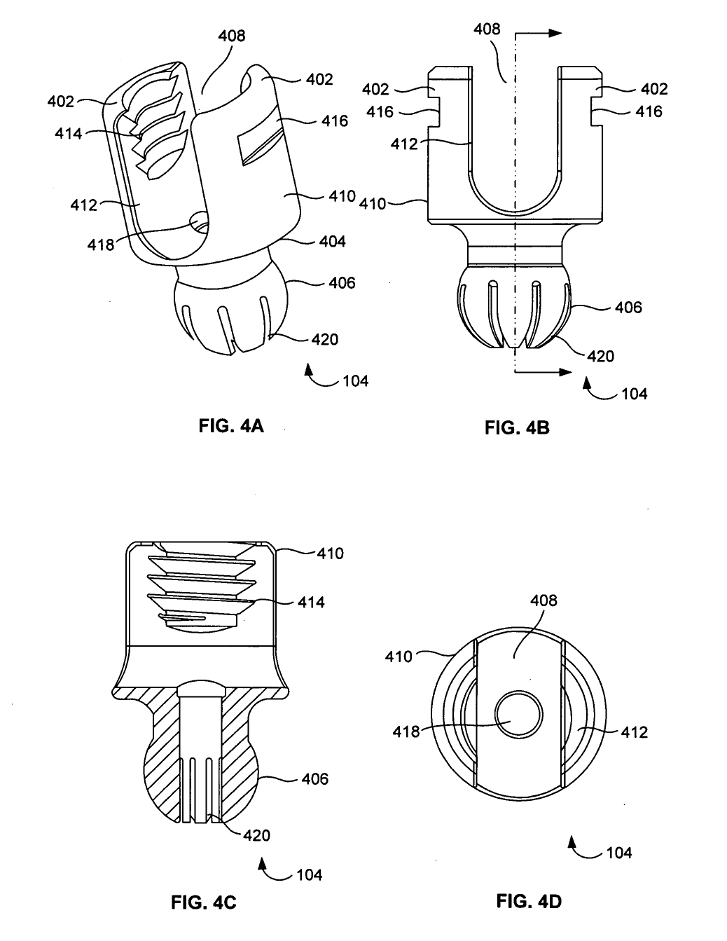

FIGS. 3E and 3F illustrate top views of the bone anchor 102 of the dynamic screw assembly 100 of FIG. 3B in the first position and the second position, respectively, according to an embodiment herein. With reference to FIGS. 3A through 3F, the open concave head 302 of the bone anchor 102 may be angled to accept the bumper(s) 106. FIGS. 4A through 4D illustrate a perspective view, a front view, a cross-sectional view, and a top view, respectively, of the coupling member 104 of the dynamic screw assembly 100 of FIGS. 1A through 1C according to an embodiment herein. With reference to FIGS. 4A through 4D, the coupling member 104 may be embodied as a screw head between the bone anchor 102 and the rod 108.

The coupling member 104 includes a pair of arms 402, an inwardly curved bottom portion 404, a bulbous end 406, and a U-shaped slot 408. The arms 402 further include an outer wall 410 and an inner wall 412. The inner wall 412 includes threads 414 to engage the blocker 204 (of FIGS. 2A through 2C). The outer wall 410 of the arms 402 includes an indent feature 416. The coupling member 104 also has an opening 418 through the middle of the spherical portion 404, which extends through the bottom of the bulbous end 406.

The bulbous end 406 includes channels 420. The U-shaped slot 408 is positioned between the arms 406 to receive the rod 108 (of FIGS. 2A through 2C). The indent feature 416 on the outer wall 410 of the coupling member 104 may be configured for various instruments (not shown) to manipulate the bone anchor 102 (of FIGS. 3A through 3F) during surgery. The channels 420 of the bulbous end 406 allow the coupling member 104 to be secured to the bone anchor 102 through expansion of the bulbous end 406 into the inner portion 306 of the open concave head 302 of the bone anchor 102 (of FIGS. 3A through 3F). The opening 418 receives the saddle connection 202 to be fixed firmly to the bone anchor 102.

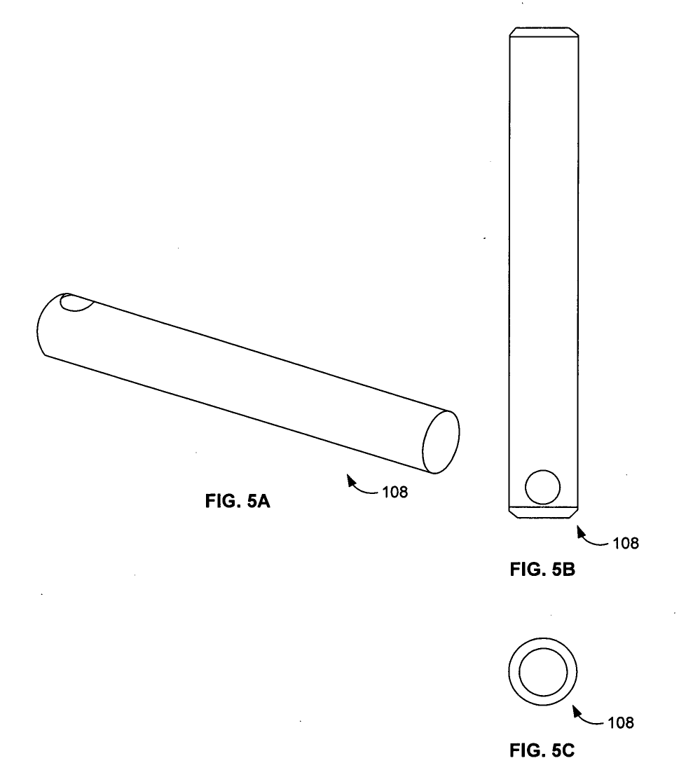

FIGS. 5A through 5C illustrate a perspective view, a front view, and a top view, respectively, of a rod 108 of the dynamic screw assembly 100 of FIGS. 1A through 1C according to an embodiment herein. The rod 108 may be a longitudinal member connecting the coupling member 104 and the saddle connection 202. The rod 108 is positioned longitudinally in the U-shaped slot 408 of the coupling member 104 (of FIGS. 4A through 4D). The rod 108 may provide a torsional movement to correct a spinal displacement and a curvature.

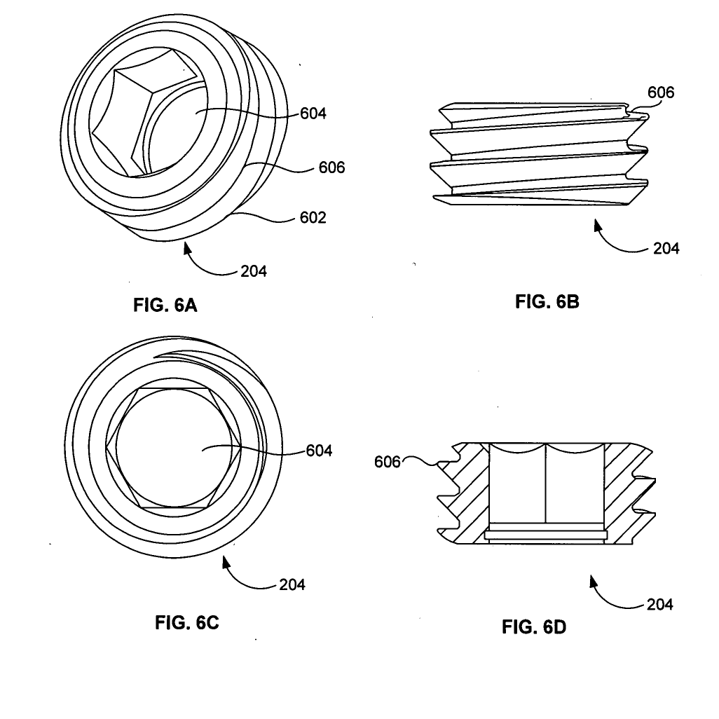

FIGS. 6A through 6D illustrate a perspective view, a front view, a top view, and a cross-sectional view, respectively, of the blocker 204 of the dynamic screw assembly 100 of FIGS. 1A through 1C according to an embodiment herein. With reference to FIGS. 6A through 6D, the blocker 204 is the securing member between the rod 108 and the coupling member 104 (of FIGS. 2A through 2C). The blocker assembly 204 includes an outer cylindrical parameter 602 and an aperture (e.g., hexagonal in one embodiment) 604 in the middle. The outer cylindrical parameter 602 includes threads 606 to engage the threads 414 of the coupling member 104, and then exerts a downward force on the rod 108 that pushes down onto the saddle connection 202 (of FIGS. 2A through 2C) effectively locking the dynamic screw assembly 100.

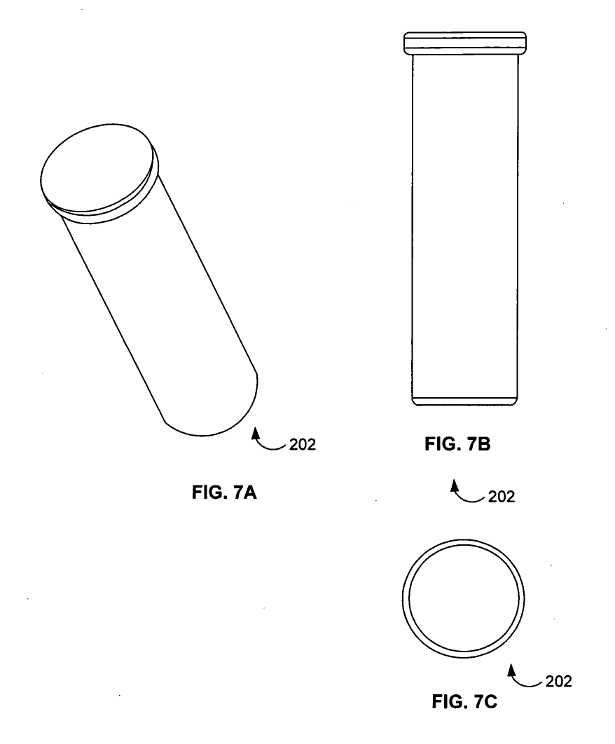

FIGS. 7A through 7C illustrate a perspective view, a front view, and a top view, respectively, of the saddle connection 202 of the dynamic screw assembly 100 of FIGS. 1A through 1C according to an embodiment herein. With reference to FIGS. 7A through 7C, the saddle connection 202 may be a longitudinal member placed along a vertical axis through the center opening 418 of the coupling member 104 (of FIGS. 4A through 4D) to prevent the coupling member 104 from disengaging the bone anchor 102 and limit angulation.

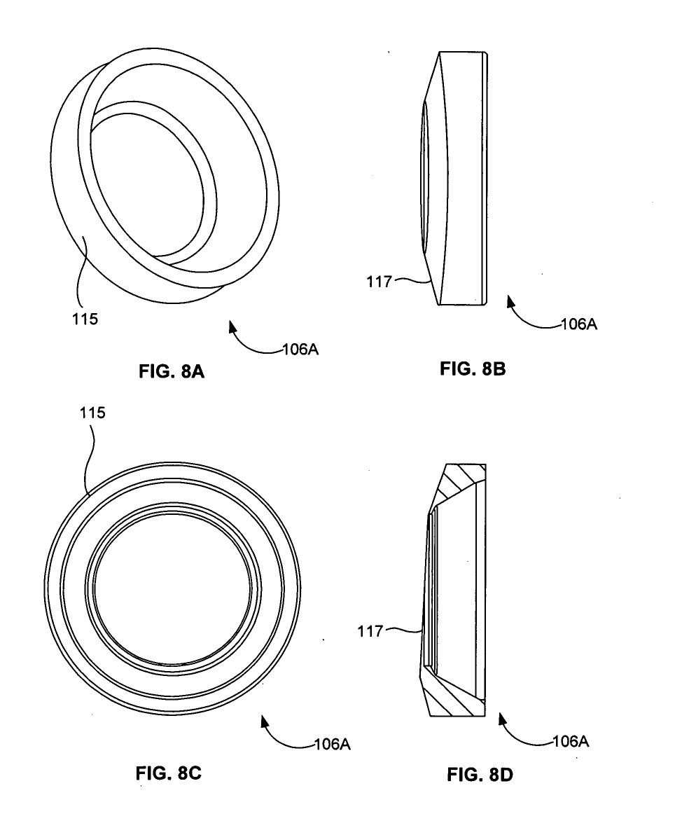

FIGS. 8A through 8D illustrate a perspective view, a front view, a top view, and a cross-sectional view, respectively, of the biased bumper 106 of the dynamic screw assembly 100 of FIGS. 1A through 1C according to a first embodiment herein. In this embodiment, the biased bumper 106A is configured as a bowl-shaped mechanism 115 with an open top and bottom. The bottom 117 of the biased bumper 106 may be beveled to provide the biasing effect.

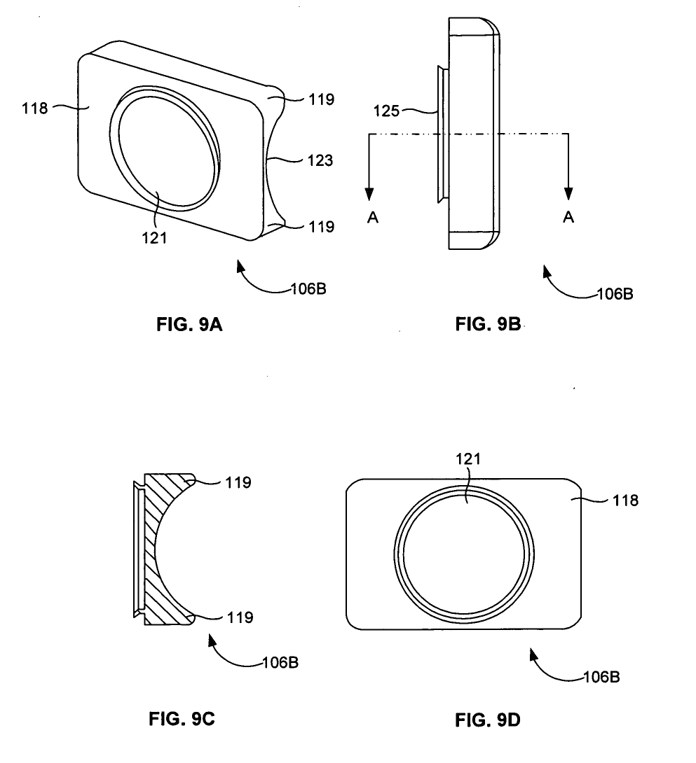

FIGS. 9A through 9D illustrate a perspective view, a front view, a cross-sectional view, and a top view, respectively, of the biased bumper 106 of the dynamic screw assembly 100 of FIGS. 1A through 1C according to a second embodiment herein. In this embodiment, the biased bumper 106B comprises a generally flat top portion 118 with a central bore 121 having a raised surface 125 extending outwardly from the top portion 118. A curved bottom portion 123 of the bumper 106B is defined by opposed curved legs 119, which provides the biasing effect.

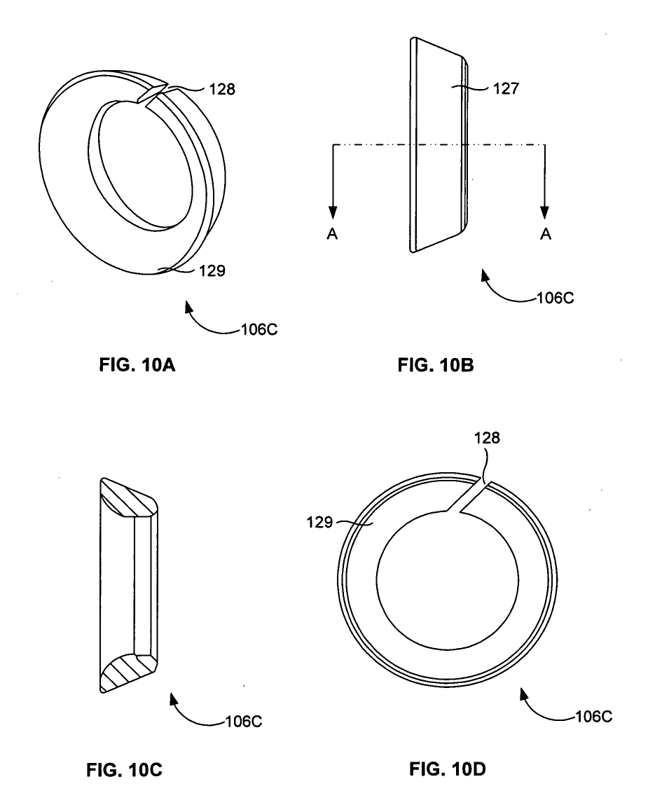

FIGS. 10A through 10D illustrate a perspective view, a front view, a cross-sectional view, and a top view, respectively, of the biased bumper 106 of the dynamic screw assembly 100 of FIGS. 1A through 1C according to a third embodiment herein. In this embodiment, the biased bumper 106C is configured as a bowl-shaped mechanism 127 with an open top and bottom. A slot 128 is included in the mechanism 127. The upper surface 129 of the biased bumper 106C may be angled to provide the biasing effect.

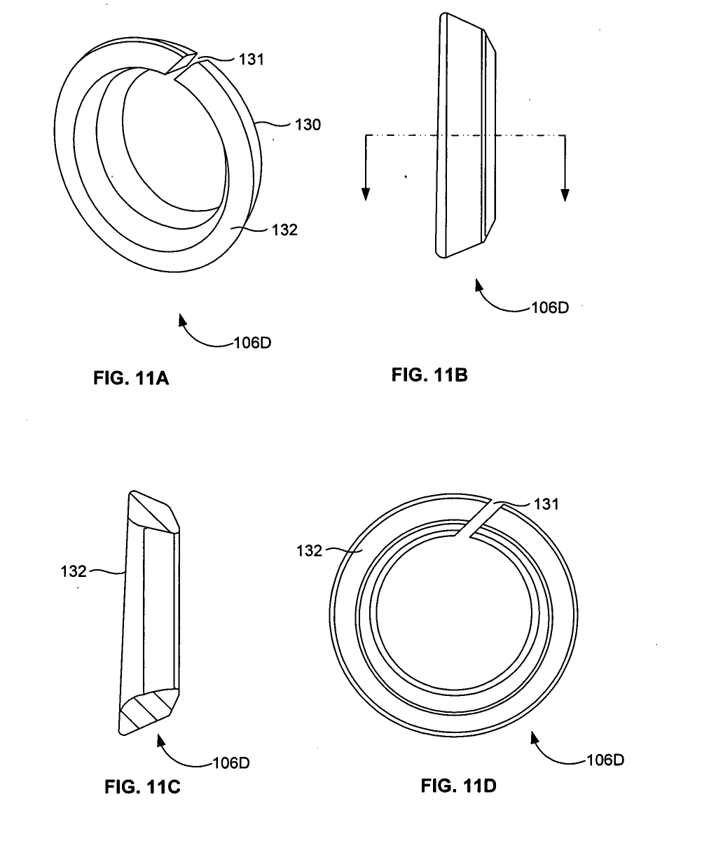

FIGS. 11A through 11D illustrate a perspective view, a front view, a cross-sectional view, and a top view, respectively, of the biased bumper 106 of the dynamic screw assembly 100 of FIGS. 1A through 1C according to fourth embodiment herein. In this embodiment, the biased bumper 106C is configured as a bowl-shaped mechanism 130 with an open top and bottom. A slot 131 is included in the mechanism 130. The upper surface 132 of the biased bumper 106D may be beveled to provide the biasing effect.

The biased bumper(s) 106 are located between the bone anchor 102 and the coupling member 104. The biased bumper(s) 106 includes two intersecting planes, one at the top and one at the bottom. The bumper(s) 106 are designed with one or more pieces of flexible materials. In one embodiment, for a one-piece bumper, the angle of the top portion of the bumper 106 and the other angle created by the bone anchor 102 generate the resultant angle. The resultant angle, created by these two components (e.g., the top portion of the bumper 106 and the bone anchor 102) may be at least one of a zero degree angle or an angle that is the sum of both angles. In an alternative embodiment, for stacked bumpers, the accumulated angles between the bumpers 106 and the bone anchor 102 determines the resultant angle based on the orientation of the bumpers 106 with respect to one another. At least one of the stacked bumpers 106 may include a slot.

The embodiments herein provide a dynamic screw assembly 100 with a biased bumper mechanism 106 that supports for dynamic stabilization. The biased bumper mechanism 106 of the dynamic screw assembly 100 assists a surgeon to adjust the angulation of the coupling member 104 to a desired location in the dynamic screw system 100 making it flexible for non-fusion applications. The bumper mechanism also allows the bone anchor 102 to be inserted in any direction and later to be adjusted to a final position, thus helping to achieve a desired polyaxial dynamism regardless of the insertion angle of the implant assembly 100. The dynamic screw assembly 100 provides both translating in different directions and rotating movements to increase the moment arm.

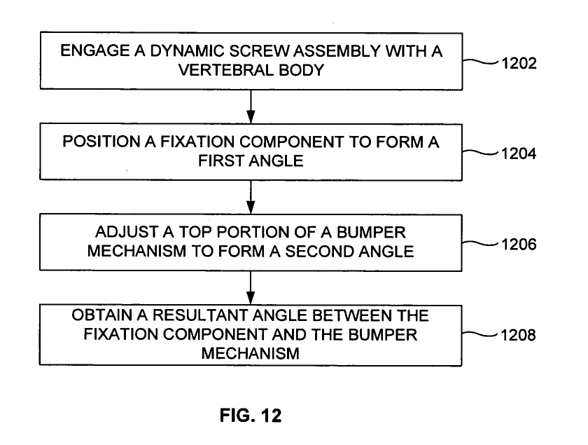

FIG. 12, with reference to FIGS. 1A through 11D, is a flow diagram illustrating a method of inserting a dynamic screw assembly in a vertebral body according to an embodiment herein. In step 1202, the dynamic screw assembly is engaged with the vertebral body. The dynamic screw assembly includes a screw head (e.g., the coupling member 104 of FIGS. 1A-C), a bumper mechanism (e.g., the biased bumper(s) 106 of FIGS. 1A-C), a fixation component (e.g., the bone anchor 102 of FIGS. 1A-C), a saddle connection (e.g., the saddle connection 202 of FIGS. 2A-C), a longitudinal member (e.g., the rod 108 of FIGS. 1A-C) and a blocker (e.g., the blocker 204 of FIGS. 2A-C).

The screw head further includes a pair of diametrically opposed arms (e.g., the pair of arms 402 of FIGS. 4A-4B), a slot (e.g., the U-shaped slot 408 of FIGS. 4A-4B, FIG. 4D) between the arms, an inwardly curved bottom portion (e.g., the inwardly curved bottom portion 404 of FIG. 4A), an outwardly protruding and expandable bulbous end (e.g., the bulbous end 406 of FIGS. 4A-4C) extending from the inwardly curved bottom portion 404 and an opening (e.g., the opening 418 FIG. 4A, FIG. 4D) positioned through the bulbous end.

The bumper mechanism (e.g., the biased bumper(s) 106 of FIGS. 1A-1C) adjacent to the screw head adjusts an angle of the screw head to a desired location in the dynamic screw assembly. The fixation component (e.g., the bone anchor 102 of FIGS. 1A-1C) is coupled to the bumper mechanism. The saddle connection (e.g., the saddle connection 202 of FIGS. 2A-2C) is positioned in the opening and engages the screw head and the fixation component. The longitudinal member (e.g., the rod 108 of FIGS. 1A-1C) is positioned in the slot. The blocker (e.g., the blocker 204 of FIGS. 2A-2C) is coupled to the screw head and the longitudinal member.

In step 1204, the fixation component is positioned to form a first angle. In step 1206, a top portion of the bumper mechanism is adjusted to form a second angle. In step 1208, a resultant angle between the fixation component and the bumper mechanism is obtained. The resultant angle may be at least one of a zero degree angle or a summation of the first angle and the second angle. The resultant angle may be obtained based on an orientation of the bumper mechanism being stacked. The resultant angle may be an accumulation of the first angle of the bumper mechanism and the second angle of the fixation component.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A dynamic screw assembly comprising:
a screw head comprising:
a pair of diametrically opposed arms;
a slot between said arms;
an inwardly curved bottom portion;
an outwardly protruding and expandable bulbous end extending from said inwardly curved bottom portion; and
an opening positioned through said bulbous end;
a bumper mechanism adjacent to said screw head that adjusts an angle of said screw head to a desired location in said dynamic screw assembly;
a fixation component coupled to said bumper mechanism;
a saddle connection positioned in said opening and engaging said screw head and said fixation component;
a longitudinal member positioned in said slot; and
a blocker coupled to said screw head and said longitudinal member.

2. The dynamic screw assembly of claim 1, wherein said bumper mechanism comprises any of a one-piece bumper and a stacked bumper.

3. The dynamic screw assembly of claim 2, wherein said one-piece bumper generates a resultant angle, said resultant angle is at least one of a zero degree angle or a sum of an angle by said one-piece bumper and said fixation component.

4. The dynamic screw assembly of claim 2, wherein said stacked bumper generates a resultant angle, and wherein said resultant angle is an accumulated angle between said stacked bumper and said fixation component.

5. The dynamic screw assembly of claim 1, wherein said bumper mechanism limits an angulation of said screw head based on an orientation of said bumper mechanism with respect to said fixation component.

6. The dynamic screw assembly of claim 1, wherein said fixation component comprises an open concave head and a threaded end, wherein said open concave head of said fixation component contacts said bumper mechanism.

7. The dynamic screw assembly of claim 6, wherein said open concave head of said fixation component comprises:
an inner portion that receives said bulbous end of said screw head, wherein said bulbous end is positioned opposite to said pair of diametrically opposed arms;
a hole; and
an outer portion comprising grooves.

8. The dynamic screw assembly of claim 7, wherein said hole of said fixation component engages said saddle connection.

9. An apparatus for dynamic spinal stabilization, said apparatus comprising:
at least one bumper comprising flexible material and composed of two intersecting planes, wherein said at least one bumper adjusts an insertion angle of said apparatus to a desired location based on an orientation of said at least one bumper;
a bone anchor having an open concave head and a threaded end, wherein said open concave end engages said at least one bumper;
a coupling member comprising:
a first portion comprising a pair of arms that are diametrically opposed;
a U-shaped slot positioned between said pair of arms;
an inwardly curved bottom portion;
a second portion having a an outwardly protruding and expandable bulbous end extending from said inwardly curved bottom portion configured to engage said open concave head of said bone anchor; and
an opening positioned between said first portion and said second portion;
a saddle connection that engages said opening of said coupling member, said saddle connection being coupled to said bone anchor;
a rod coupled to said U-shaped slot; and
a threaded blocker that engages said pair of arms of said coupling member and secures said rod in said coupling member.

10. The apparatus of claim 9, wherein said open concave head of said bone anchor further comprises:
an inner portion that receives said bulbous end of said coupling member;
a hole that engages said saddle connection; and
an outer portion comprising grooves.

11. The apparatus of claim 9, wherein said pair of arms comprises an outer wall and an inner wall, said outer wall comprising an indent feature and said inner wall comprising threads.

12. The apparatus of claim 11, wherein said at least one bumper comprises any of at least a one-piece bumper and a stacked bumper, wherein said stacked bumper comprises a slot and generates a resultant angle, and wherein said resultant angle is an accumulated angle between said stacked bumper and said bone anchor.

13. The apparatus of claim 12, wherein said one-piece bumper generates a resultant angle, and wherein said resultant angle is at least one of a zero degree angle or a sum of an angle by said one-piece bumper and said bone anchor.

14. A method of inserting a dynamic screw assembly in a vertebral body, said method comprising:
engaging said dynamic screw assembly with said vertebral body, said dynamic screw assembly comprising:
a screw head comprising:
a pair of diametrically opposed arms;
a slot between said arms;
an inwardly curved bottom portion;
an outwardly protruding and expandable bulbous end extending from said inwardly curved bottom portion; and
an opening positioned through said bulbous end;
a bumper mechanism adjacent to said screw head that adjusts an angle of said screw head to a desired location in said dynamic screw assembly;
a fixation component coupled to said bumper mechanism;
a saddle connection positioned in said opening and engaging said screw head and said fixation component;
a longitudinal member positioned in said slot; and
a blocker coupled to said screw head and said longitudinal member;
positioning said fixation component to form a first angle;
adjusting a top portion of said bumper mechanism to form a second angle; and
obtaining a resultant angle between said fixation component and said bumper mechanism, wherein said resultant angle is at least one of a zero degree angle or a summation of said first angle and said second angle.

15. The method of claim 14, further comprising obtaining said resultant angle based on an orientation of said bumper mechanism being stacked, wherein said resultant angle is an accumulation of said first angle of said bumper mechanism and said second angle of said fixation component.

16. The method of claim 15, wherein said stacked bumper mechanism generates a resultant angle, and wherein said resultant angle is an accumulated angle between said stacked bumper mechanism and said fixation component.

17. The method of claim 14, wherein said bumper mechanism limits an angulation of said screw head based on an orientation of said bumper mechanism with respect to said fixation component.

18. The method of claim 14, wherein said fixation component comprises an open concave head and a threaded end, wherein said open concave head of said fixation component contacts said bumper mechanism.

19. The method of claim 14, wherein said open concave head of said fixation component comprises:
an inner portion that receives said bulbous end of said screw head, wherein said bulbous end is positioned opposite to said pair of diametrically opposed arms;
a hole; and
an outer portion comprising grooves.

20. The method of claim 19, wherein said hole of said fixation component engages said saddle connection.
